(19) United States
(12) Patent Application Publication
(10) Pub. No.: US 2007/0214006 A1
(43) Pub. Date: Sep. 13, 2007

(54) Method and system for producing performance reports for payors

(76) Inventors: David Wayne Duckert, Menomonee Falls, WI (US); James Steven Cincotta, New Berlin, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/371,055

(22) Filed: Mar. 8, 2006

(51) Int. Cl. G06Q 10/00; A61B 5/00

(52) U.S. Cl. 705/2; 600/300

(57) ABSTRACT

The method and system includes producing performance reports for payors that require performance evidence. The method and system collects a set of clinical data from the patient, combines the set of remote patient data with a set of EMR data, de-identifies this data, and produces a set of reports including compliance reports and outcomes reports, which can be tailored to a payor's needs, and further delivered to the payor. The method may be implemented as software to be executed on the system.

### FIELD OF THE INVENTION

The invention relates to the field of remote monitoring. More particularly, the invention relates to the field of performance measurement in remote monitoring.

### BACKGROUND OF THE INVENTION

For a variety of reasons, monitoring of chronically ill patients in a remote, non-hospital environment will become more common in the near future. The clinical data collected, for example, blood pressure, weight, self-assessments, etc., is transmitted back to a caseworker or clinician who can provide early intervention to prevent re-hospitalizations. By monitoring patients remotely, costly re-hospitalization events can be avoided and the overall cost of managing the disease can be reduced.

One of the barriers to implementing home monitoring systems is measuring their benefit. The benefit of these systems can be measured in several ways. The first way is measured in terms of clinical outcomes, where payors may set benchmarks or target goals for re-hospitalization rates, mortality rates, or other outcome-type metrics. The second way is measured in terms of compliance, where payors may demand that chronic disease management programs are being implemented uniformly and in accordance with clinically proven guidelines. Metrics related to frequency of contact, types of interventions, medications prescribed, etc. might be used to measure a program's success.

Payors including the Centers for Medicare & Medicaid Services (CMS), may institute “Pay for Performance” requirements, thereby requiring providers to provide evidence of performance. This may include clinical outcomes and compliance to published guidelines. Current chronic disease monitoring systems do not provide this type of performance evidence.

### SUMMARY OF THE INVENTION

The method and system includes producing performance reports for payors that require performance evidence. The method and system collects a set of clinical data from the patient, combines the set of remote patient data with a set of EMR data, de-identifies this data, and produces a set of reports including compliance reports and outcomes reports, which can be tailored to a payor's needs, and further delivered to the payor. The method may be implemented as software to be executed on the system.

In one aspect of the present invention, a method of producing a set of performance reports for a payor comprises collecting a set of remote patient data from a patient, combining the set of remote patient data with a set of electronic medical record data to produce a set of combined data, de-identifying the set of combined data to produce a set of de-identified data and compiling the set of de-identified data in a performance database to produce the set of performance reports. The set of remote patient data may also be directly collected in the set of electronic medical record data. The method further includes storing the set of de-identified data in the performance database before the compiling step. This method further comprises formatting the set of performance reports to comply with a set of payor specifications, delivering the set of performance reports to the payor, and the de-identifying step includes redacting all patient references and personal information from the set of combined data. This set of performance reports may also include a compliance report that identifies a compliance level with a set of established treatment protocols, or that tracks a level of compliance over time, and a clinical outcomes report that may be a comparative clinical outcome report or a tracking clinical outcome report.

In yet another aspect of the present invention, a system for producing a set of performance reports for a payor comprises a remote sensing system configured to collect a set of remote patient data from a patient, a storage media for storing a computer application and a processing unit coupled to the remote sensing system and the storage media, the processing unit configured to execute the computer application, and further configured to receive the set of patient data from the remote sensing system, wherein when the computer application is executed, the set of remote patient data is combined with a set of electronic medical record data from an electronic medical record database to produce a set of combined data, the set of combined data is de-identified to produce a set of de-identified data, and the set of de-identified data is compiled in a performance database to produce a set of performance reports. In this system the set of remote data may be directly collected in the set of electronic medical record data, and the set of de-identified data is stored in the performance database before the set of de-identified data is compiled. The set of performance reports are formatted to comply with a set of payor specifications and the set of performance reports are delivered to the payor and the set of combined data is de-identified by redacting all patient references and personal information from the set of combined data. This set of performance reports may include a compliance report that identifies a compliance level with a set of established treatment protocols or a compliance report that tracks a level of compliance over time, and the set of performance reports may include a clinical outcomes report which may be a comparative clinical outcome report or a tracking clinical outcome report.

### BRIEF DESCRIPTION OF THE DRAWINGS

FIG. 1 illustrates a flow chart of a method in accordance with an embodiment of the present invention.

FIG. 2 illustrates a graphical representation of a method in accordance with an embodiment of the present invention.

FIG. 3 illustrates a block diagram of a system in accordance with an embodiment of the present invention.

### DETAILED DESCRIPTION OF THE INVENTION

The method and system combines remotely acquired patient data with available electronic medical records data and de-identifies this combined data before storing the de-identified data in a performance database. The method and system compiles the stored de-identified database to produce a set of performance reports for a particular payor. Payors may include government agencies, such as the Centers for Medicare and Medicaid Systems (CMS), private insurance companies or self-insured firms, or third party such as auditing firms. The set of performance reports are formatted to comply with the particular payor's specifications, and delivered to the payors.

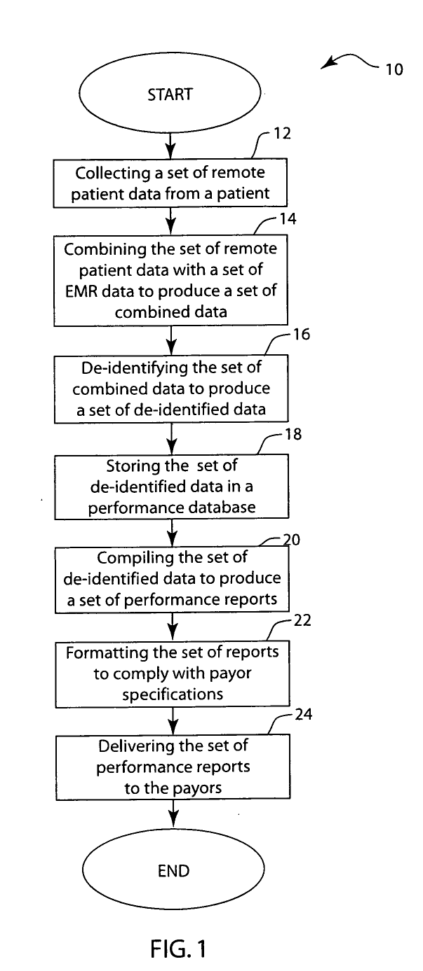

FIG. 1 depicts such a reporting method 10. In step 12, a set of remote patient data is collected from a patient. This set of remote patient data can be any physiological or self-assessment data that is capable of being collected remotely and desired to be included in a set of performance reports by a payor. In step 14, the set of remote patient data is combined with a set of electronic medical record (EMR) data to produce a set of combined data. In further embodiments, the remote patient data can be directly collected and captured in the EMR in step 12, eliminating the need for step 14. The EMR data includes any known inpatient or outpatient electronic medical records previously collected and stored for the remotely monitored patient. In step 16, the set of combined data is de-identified to produce a set of de-identified data. De-identification includes removing any patient references from the combined data, including any personal information such as social security numbers, patient name, patient address or other such information. In step 18, the set of de-identified data is stored in a performance database, and in step 20, the set of de-identified data is compiled to produce the set of performance reports.

Still referring to FIG. 1, in step 22, the set of performance reports are formatted to comply with payor specifications. This formatting step includes creating different types of performance reports, as well as simply formatting the reports in an electronic or paper format in compliance with payor specifications. The two main types of performance reports that are compiled and formatted for the payor include compliance reports and clinical outcomes reports. One type of compliance report identifies the level of compliance of patient populations, health care institutions, or clinicians have with established treatment protocols. These reports assist in identifying which particular populations, health care institutions, or clinicians are not providing treatment according to the commonly accepted protocols. Another kind of compliance report tracks the level of compliance for a particular population, health care institution, or clinician over time, thereby indicating the level of change experienced by a population, health care institution, or clinician. One type of clinical outcomes report compares the clinical outcomes of a particular patient with other health care institutions, populations, clinicians, or treatment protocols. Such outcomes may be measured as re-hospitalization rate, mortality rate, quality of life indices, or patient satisfaction survey scores. A second type of clinical outcomes report tracks a particular population, health care institution, or clinician over time, thereby following the level of changed experience by a population, health care institution or clinician. Finally, in step 24, the set of performance reports are delivered to the payors. This delivery step may be effectuated by any electronic means currently known such as over a network, or by hard copy.

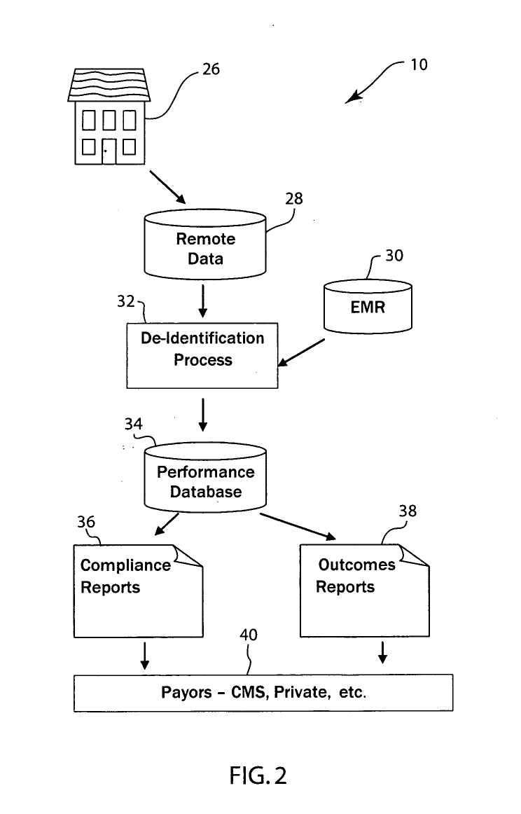

Referring to FIG. 2, a block diagram of the reporting method 10 is depicted. Here, remote patient data is collected from a patient in a home environment 26, and that remote data 28 is combined with EMR data 30, and a de-identification process 32 is conducted on this combined data. A performance database 34 stores this de-identified data until the data is compiled into a performance report. As stated previously, the performance report may include either a compliance report 36 and/or an outcome report 38, and is delivered to a payor 40. Again, such payors that may require such performance reports will include government agencies such as CMS, private insurance companies, self-insured firms, or third parties, such as auditing firms. These performance reports may be aggregated to create regional or national level compliance and outcomes reporting. Such reports may be used by researchers to suggest improvements to disease treatment guidelines, as well as by internal providers to monitor disease management program improvements and changes.

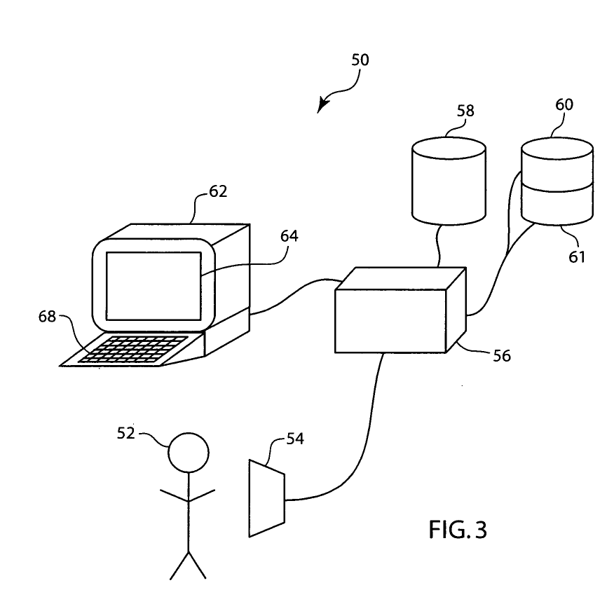

It should be understood that the method may be implemented as software and run on an appropriate reporting system 50 including a storage medium 58, a processor 56, an electronic device 62 such as a computer, laptop, PDA, or other similar device, and be compatible with the remote sensing system 54 as well as appropriate databases 60, 61. FIG. 3 illustrates an embodiment of this system.

Referring now to FIG. 3, the computer code embodying the software is stored in the storage media 58. The remote sensing system 54 collects the remote patient data form the patient 52 and sends the remote patient data to the processor 56. Executing the computer code, the processor 56 accesses an EMR database 60 for the patient's electronic medical record, and combines patient's EMR data with the remote patient data, and stores that combined data in the performance database 61. When a user requires a performance report, the processor 56 retrieves the stored combined data from the performance database 61 and compiles the combined data to produce the performance report. The performance report may then be displayed on a graphical user interface 64 of the electronic device 62. The electronic device 62 further includes an input/output device 68 so that a user 52 may manipulate the performance report, save or forward the report, or even request a new report with different parameters or a different type of report. It should be noted that the performance report delivered to the electronic device 62 may be directly viewed by a payor in some embodiments, or by a user of the software that is producing the performance reports for a payor. In other words, the method and system may be implemented specifically for a payor and operated by that payor, or may be operated by a third party or clinical staff preparing such performance reports for the payor. Furthermore, the report may also be provided in hardcopy or stored as an electronic file or a disk.

This method and system provides the advantages of having automatic generation of pay for performance reports, which will save time and money. Rather than compiling this type of information manually, the reports will be automatically generated and sent to the payors in the proper format at the correct time, which will result in cost saving for care providers of chronic disease care.

The present invention has been described in terms specific embodiments incorporating details to facilitate the understanding the principles of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirited scope of the invention.

1. A method of producing a set of performance reports for a payor, the method comprising:
collecting a set of remote patient data from a patient;
combining the set of remote patient data with a set of electronic medical record data to produce a set of combined data;
de-identifying the set of combined data to produce a set of de-identified data; and
compiling the set of de-identified data in a performance database to produce the set of performance reports.

2. The method as claimed in claim 1, wherein the set of remote patient data is directly collected in the set of electronic medical record data.

3. The method as claimed in claim 1, farther comprising storing the set of de-identified data in the performance database before the compiling step.

4. The method as claimed in claim 1, further comprising formatting the set of performance reports to comply with a set of payor specifications.

5. The method as claimed in claim 1, further comprising delivering the set of performance reports to the payor.

6. The method as claimed in claim 1, wherein the de-identifying step includes redacting all patient references and personal information from the set of combined data.

7. The method as claimed in claim 1, wherein the set of performance reports include a compliance report.

8. The method as claimed in claim 7, wherein the compliance report identifies a compliance level with a set of established treatment protocols.

9. The method as claimed in claim 7, wherein the compliance report tracks a level of compliance over time.

10. The method as claimed in claim 1, wherein the set of performance reports includes a clinical outcomes report.

11. The method as claimed in claim 10, wherein the clinical outcomes report is a comparative clinical outcome report.

12. The method as claimed in claim 10, wherein the clinical outcomes report is a tracking clinical outcome report.

13. A system for producing a set of performance reports for a payor, the system comprising:
a remote sensing system configured to collect a set of remote patient data from a patient;
a storage media for storing a computer application; and
a processing unit coupled to the remote sensing system and the storage media, the processing unit configured to execute the computer application, and further configured to receive the set of patient data from the remote sensing system,
wherein when the computer application is executed, the set of remote patient data is combined with a set of electronic medical record data from an electronic medical record database to produce a set of combined data, the set of combined data is de-identified to produce a set of de-identified data, and the set of de-identified data is compiled in a performance database to produce a set of performance reports.

14. The system as claimed in claim 13, wherein the set of remote data is directly collected in the set of electronic medical record data.

15. The system as claimed in claim 13, wherein the set of de-identified data is stored in the performance database before the set of det-identified data is compiled.

16. The system as claimed in claim 13, wherein the set of performance reports are formatted to comply with a set of payor specifications.

17. The system as claimed in claim 13, wherein the set of performance reports are delivered to the payor.

18. The system as claimed in claim 13, wherein the set of combined data is de-identified by redacting all patient references and personal information from the set of combined data.

19. The system as claimed in claim 13, wherein the set of performance reports include a compliance report.

20. The system as claimed in claim 19, wherein the compliance report identifies a compliance level with a set of established treatment protocols.

21. The system as claimed in claim 19, wherein the compliance report tracks a level of compliance over time.

22. The system as claimed in claim 13, wherein the set of performance reports includes a clinical outcomes report.

23. The system as claimed in claim 22, wherein the clinical outcomes report is a comparative clinical outcome report.

24. The system as claimed in claim 22, wherein the clinical outcomes report is a tracking clinical outcome report.

25. A method of providing a set of performance reports to a payor, the method comprising:
collecting a set of remote patient data from a patient;
combining the set of remote patient data with a set of electronic medical record data to produce a set of combined data;
redacting the set of combined data to produce a set of de-identified data;
storing the set of de-identified data in a performance database;
compiling the set of de-identified data to produce the set of performance reports;
formatting the set of performance reports to comply with a set of payor specifications; and
delivering the set of performance reports to the payor.
